(19) United States
(12) Patent Application Publication
(10) Pub. No.: US 2006/0111723 A1
(43) Pub. Date: May 25, 2006

(54) Orthopedic medical device with unitary components

(76) Inventors: Robert J. Chapolini, Phoenix, MD (US); Russell D. Geise, Allentown, PA (US)

Correspondence Address: William J Dundren, 734 LaRue Road, Millersville, MD, 21108, US

(21) Appl. No.: 10/529,345

(22) PCT Filed: Sep. 25, 2003
(86) PCT No.: PCT/US03/29983

§ 371 Date: Nov. 16, 2005

Related U.S. Application Data
(60) Provisional application No. 60/413,458, filed on Sep. 26, 2002.

(51) Int. Cl. A61B 17/16

(52) U.S. Cl. 606/80

(57) ABSTRACT

A cordless medical apparatus suitable for use in orthopedic medical applications is disclosed. The medical device includes a disposable sterile housing having a sleeve, said sleeve being configured to receive an unsterilized motor assembly. The invention further includes a sterile packaging system for enclosing the sterile housing and inserting a non-sterile component in the sterile housing.

### (B) CROSS-REFERENCE TO RELATED APPLICATIONS

Not applicable.

### (C) STATEMENT REGARDING FEDERALLY SPONSORED RESEARCH

Not applicable.

### (D) BACKGROUND OF THE INVENTION

(D1) Field of the Invention

The invention relates to a battery-powered medical device for bone tissue in a living body. The term “tissue” as used herein, refers, but is not limited to dense structures such as bone and other calcified structures. The primary purposes of a medical device such as a drill are its use in fixing or stabilizing two pieces of bone in relation to each other, and grinding or shaping bone. The drill, equipped with a coring bit or trephine or the like, may also be used for diagnosing a suspected pathologic condition involving the tissue, and/or for the purpose of completely removing all diseased tissue, thus effecting treatment of the disease. Also, a drill may be used in therapeutic procedures, e.g., removing a portion of bone for its use in a bone graft.

The present invention also relates to an apparatus and method for packaging devices having both a sterile component and a non-sterile component, a circumstance that is common to the medical industry, among others. For example, using a packaging system of the present invention, a battery powered surgical drill (the sterile component) may be assembled with a removable, rechargeable battery (the non-sterile component) within the confines of a clean or sterile room or area.

(D2) Description of Related Art

The use of motorized devices for therapeutic and medical purposes is very well known in the medical arts. Various types of motorized or pneumatic devices are available for various particular applications. For example, drills and saws are used for the examination and repair in many orthopedic surgical procedures. Coring devices may be used to biopsy or sample bone marrow. Most of these devices are corded, that is, they include a power cord that extends from the housing of the device to a source of electricity, or they include a pneumatic cord that extends from the housing of the device to a source of pressurized gas (typically nitrogen). It would be highly desirable to provide an untethered or cordless device.

Cordless surgical devices such as drills are frequently used in the operating room and avoid some of the problems associated with corded devices. Non-corded or cordless devices are typically not disposable in whole or in part. Also, these drills typically have replaceable battery packs which must be sterilized before use. Steam sterilization can substantially reduce the life expectancy of the battery pack. For example, a battery powered surgical drill typically has a removable, rechargeable battery pack. Although many of these battery packs can be steam sterilized, battery life can be extended by eliminating the sterilizing process and loading the battery into the drill aseptically. It would be highly desirable to provide a cordless, disposable medical device.

Medical and surgical devices continue to become more sophisticated and expensive. Typically, these instruments must be sterilized before use. Instruments that are reusable must be made very durable to withstand repeated sterilizing cycles, adding considerable manufacturing cost In order to reduce the cost of these instruments, some manufacturers have provided inexpensive sterile sleeves that completely cover the reusable portion, thus eliminating the need for sterilizing the reusable portion.

Motorized medical devices provide tremendous advantages in the treatment and repair of many medical conditions, but are typically limited in their capability of repeated use and in the inability or complexity of sterilizing the device between uses. For example, the sterilization of most medical devices presents several substantial disadvantages. First, the device must be manufactured to withstand such sterilization. That is, the device must be formed of materials that will not degrade in the presence of those sterilizing agents in contemporary use. It is important to note that the grade of materials is typically not a function of the surgical procedure itself; a higher grade material is typically a function of the need to withstand repeated sterilizations.

This inherently requires that more expensive materials and manufacturing techniques be utilized and that the sterilization-degradable components be adequately isolated from or compatible with such sterilizing agents.

It would be highly desirable to provide a device having components of a grade suited to the specific surgical procedure, thereby reducing the costs associated with the device. It would also be desirable to provide a device that is fully disposable or partially disposable in order to eliminate or reduce degradation of the components subject to sterilization, and to reduce or eliminate costs associated with sterilizing the device.

Second, thorough and proper sterilization of the device requires particular care and is consequently a time-consuming operation. Of course, when the device is being sterilized, it is not available for diagnostic and therapeutic use. Because of the high cost of most motorized medical devices and the consequent lack of availability of spares, the down-time necessitated by sterilization represents lost income for the medical facility. This loss of income increases the cost of performing even simple procedures.

Third, even with the use of such effective sterilizing agents as glutaraldehyde, adequate sterilization of the device cannot be assured. This is of particular concern when the device has a working channel or other such difficult to-clean portions. Increasingly, properly sterilized devices are being associated with a pyrogenic response (inducing fever or inflammation). Furthermore, the use of such toxic materials as glutaraldehyde presents a hazard to the patient in that tissue irritations may result from inadequate flushing of the sterilizing agent from the device. Additionally, special equipment such as a ventilated hood, is required in the use of such toxic sterilization agents, thus increasing the cost of performing such procedures. It would be highly desirable to eliminate the possibility of a pyrogenic response by providing a fully disposable device, or to reduce the possibility of a pyrogenic response by providing a partially disposable device.

For some devices, the currently accepted method of sterilization involves the use of a gas sterilization procedure, typically by exposing the device to ethylene oxide gas for a period of approximately twenty-four hours. As will be recognized, this involves an extended amount of time during which the device is not available for diagnostic and therapeutic use. As with glutaraldehyde, ethylene oxide gas is extremely toxic and extremely corrosive. Therefore, exposure to personnel must be prevented and traces of the gas must be removed from the device prior to its use to prevent tissue irritation.

Finally, it would be highly desirable to provide a medical device that is always immediately capable of use. The medical devices of the prior art, whether corded or cordless, require sterilization, maintenance, cleaning, and/or special packaging prior to re-use. The cordless, disposable medical devices of the present invention avoid these complicated, expensive, and time-consuming hidden costs associated with prior art devices.

### (E) SUMMARY OF THE INVENTION

The present invention addresses and alleviates the above-mentioned deficiencies in the prior art. The invention is an untethered or cordless drill designed for orthopedic use having a removable motor and/or battery assembly that does not need to be sterilized, or the entire device (typically without the removable battery assembly) may be disposable. By eliminating the need to sterilize the device, the device may be formed of lighter, less expensive materials. Lighter components and a lighter overall device are particularly desirable in terms of its contribution to sensitivity or feel. As used wherein, sensitivity refers to the ability of the user to sense changing conditions during use. For example, a skilled surgeon can feel or sense when a drill bit passes from hard bone into the softer bone marrow.

Components of the drill, such as the housing, may be disposable. A disposable configuration of the device may be configured for use with bits and chucks and the like that can be disposable or repeatedly sterilized, as may be optimum for a particular operation or process.

The packaging system of the present invention also permits the operator to exchange components of the device during the surgical procedure. For example, it may be desirable to exchange a first battery/motor assembly with a second battery/motor assembly during the operation. Since each of the battery/motor assemblies may be unsterilized, the packaging system configured according to the invention permits the exchange of unsterilized assemblies within a sterile field.

The present invention will foster more innovation in the design of semi-disposable surgical and medical devices and instruments. These designs may combine sophisticated reusable motorized, electronic, and/or mechanical elements adapted to communicate with or engage a sterile device. For example, the hollow interior of a handle or grip of a disposable drill may be shaped (i.e., adapted) to receive a reusable, rechargeable battery.

The present invention will also foster innovative processes in the use and processing of devices enclosed in packaging formed according to the present invention. For example, the process for inserting a non-sterile component into a sterile device in preparation for a surgical procedure will be greatly simplified using packaging according to the present invention. In this way, the coordinated efforts between a gloved personnel and non-gloved personnel can be streamlined, thereby reducing the chance of contamination.

In some embodiments of the present invention, all portions of the medical device that contact the patient or a patient's body fluid, i.e., become contaminated, are disposable. All those components that are re-usable are housed and protected from contact with the patient or body fluid, and may be easily separated from the disposable/contaminated components.

These, as well as other advantages of the present invention, will be more apparent from, the following description and drawings. It is understood that changes in the specific structure shown and described may be made within the scope of the claims without departing from the spirit of the invention.

The detailed description set forth below in connection with the appended drawings is intended as a description of the presently preferred embodiments of the invention, and is not intended to represent the only forms in which the present invention may be constructed or utilized. The description set forth the functions and sequence of steps for constructing and operating the invention in connection with the illustrated embodiments. It is to be understood, however, that the same or equivalent functions and sequences may be accomplished by different embodiments that are also intended to be encompassed within the spirit and scope of the invention.

### (F) DESCRIPTION OF THE DRAWINGS

FIG. 1 is a side view of a orthopedic medical device according to the invention.

FIG. 2 is a cross section side view of an orthopedic medical device according to the invention.

FIG. 3 is an expanded cross section of the neck portion of a medical device according to the invention.

FIG. 4 is the cross section of FIG. 3 at A-A.

FIG. 5 is the cross section of FIG. 3 at B-B.

FIG. 6 is an expanded cross section of an alternative neck portion of a medical device according to the invention.

FIG. 7 is a cut-away perspective view illustrating the sterile package enclosed in a sterile pouch.

FIG. 8 is a top cut-away view of a non-sterile motor being loaded into a sterile drill enclosed within the sterile package.

FIG. 9 shows the sterile package in its opened position, with the combined drill enclosure and motor available to be removed by a gloved operator.

FIG. 10 is a detail view of the sterile package showing a port configuration adapted to position the sleeve of the medical device.

FIG. 11 is a cross section side view of a medical device according to the invention illustrating an exemplary coupler assembly in its open position.

FIG. 12 is a cross section side view of a medical device according to the invention illustrating an exemplary coupler assembly in its closed position.

FIG. 13 is a perspective view of an alternative embodiment of the invention, showing a medical device with unitary components.

FIG. 14 is a cross section view of the medical device of FIG. 13.

### (G) DETAILED DESCRIPTION OF THE INVENTION

The present invention is a disposable medical device for orthopedic use, wherein the medical device comprises a sterile housing, said housing having a sleeve configured to receive an unsterilized motor assembly.

The present invention is a medical device for orthopedic use wherein the medical device and/or one or more of its components are immunogen- and/or pyrogen-free. In preferred embodiments of this aspect of the invention, the medical device is a drill, preferably a drill configured and suitable for use in orthopedic procedures.

The present invention also includes a package designed and configured to maintain a medical device in an immunogen-free and/or pyrogen-free condition. In preferred embodiments of the invention, the package includes at least one port that communicates with an internal portion of the medical device. In preferred embodiments of this aspect of the invention, the port permits assembly of a non-sterile component of the medical device with a sterile component.

Some embodiments of the invention include a medical device according to the invention packaged or adapted to be packaged in a sterile enclosure, said sterile enclosure having a port that communicates with an internal portion of a sleeve of the medical device.

In some embodiments of the invention, the medical device includes a sterile housing having a sleeve configured and adapted to receive a non-sterile motor or motor assembly, a drive shaft engaged with the motor, and at least one bushing or the like to position the drive shaft in the housing. In some embodiments of the invention, a distal end of the drive shaft is adapted to engage a tool, including, but not limited to, a drill bit. In some embodiments of the invention, the drive shaft includes a distal end configured to function as a tool, e.g., the end configured as a drill bit, or shaped with cutting teeth, or the like.

In some embodiments of the invention, the medical device includes a sterile housing having a sleeve configured and adapted to receive a non-sterile motor or motor assembly, a drive shaft engaged with the motor, a spline positioned in the housing and matingly engaged to the drive shaft, and a removable neck portion of the housing, said neck having an aperture to allow the drive shaft to pass therethrough. The spline is one or more structures that engage the drive shaft parallel to its axis and typically functions to transmit torque. One of more splines may be inside or outside of the housing, inside a neck or collar portion of the housing, or outside the housing at one or both ends of the drive shaft. In a preferred embodiment of the invention, the drive shaft passes through and extends beyond the spline, even more preferably so that the spline positions and maintains one end of the drive shaft in a concentric position in relation to another end of the drive shaft. In these embodiments of the invention, the drive shaft will typically further include one or more couplings engaged with the drive shaft and for attaching or connecting a tool to the drive shaft. The coupling may be positioned on the drive shaft inside or outside of the housing. In some embodiments of the invention, the drive shaft includes a distal end configured to function as a tool, e.g., the end configured as a drill bit, or shaped with cutting teeth, or the like.

As noted above, a disposable medical device for orthopedic use comprises a sterile housing, said housing having a sleeve configured to receive an unsterilized motor assembly. In preferred embodiments of the invention, the medical device is a drill. In accordance with the present invention, the drill includes a housing having a portion configured and adapted to engage a non-sterile motor assembly. The drill also includes a drive shaft positioned in the housing, said drive shaft being configured to engage the motor assembly.

A medical device of the present invention may also comprise one or more unitary and/or sealed sub-components. In an exemplary configuration, the medical device may comprise a battery/handle component, a motor component, and a collet assembly component. In these embodiments of the invention, each of the individual sub-components may be configured to fixedly or releasably engage one or more other sub-components. In some embodiments of the invention, a collet sub-assembly or component may be replaceable and/or disposable. In some embodiments of the invention, a motor sub-component may be replaceable, disposable, fluid tight, and/or sterile tight, or combination thereof. In preferred embodiments of the invention, the handle/battery sub-component is fluid or water tight, and may be submersible.

A medical device of the present invention is preferably configured to be as lightweight as possible; to be formed using materials that do not collect pyrogens and/or immunogens, preferably using materials that are non-pyrogenic and/or non-immunogenic; to be shaped into a structure having few, fewer, or no areas that collect pyrogens or immunogens; and preferably, to be formed of materials that are recyclable.

One skilled in the art will readily recognize that a medical device, such as a drill, may also include one or more of the following: an on switch, an off switch, a forward switch, a reverse switch, a combined forward/reverse switch, a variable speed switch, or combinations thereof; a switch lock; a removable or non-removable chuck, either keyed or keyless or quick-release, either disposable or non-disposable, or combinations thereof; a removable or non-removable collet, either disposable or non-disposable, or combinations thereof; a drive shaft lock; and/or one or more single-use batteries, one or more rechargeable batteries, one or more reusable batteries, or combinations thereof. It is intended that the medical device of the present invention may be variously configured with various combinations of these elements, and/or other elements, as would be apparent to one skilled in the art.

In some embodiments of the invention, one or more of the elements described above may be incorporated into or may comprise a sub-component.

Various embodiments of a medical device and corresponding packaging are described below. A preferred embodiment includes, but is not limited to, a disposable housing; disposable, single use batteries; a re-usable motor; a cannulated drive shaft; and a disposable, sterilizable package configured to nest the medical device therein.

An alternative embodiment of a medical device and corresponding packaging include, but are not limited to, a disposable housing; one or more re-usable batteries integrated with a re-usable motor, and a disposable, sterilizable package configured to nest the medical device therein.

Yet another alternative embodiment of a medical device and corresponding packaging include, but are not limited to, a disposable housing having a port for a motor and a port for a battery; one or more re-usable batteries; a separate re-usable motor; and a disposable, sterilizable package configured to nest the medical device therein.

In accordance with the present invention, the housing of the medical device may be variously configured. Any housing that includes an internal portion, such as a sleeve or a hollow handle, is included within the present invention. In accordance with the present invention, the housing includes a port or the like that permits access to or communication with the internal portion. For example, the housing may include a hollow handle having a cap or the like that opens or is removable, allowing access to the hollow portion of the handle. In this embodiment of the invention, the sleeve or hollow handle have a port that allows access to the interior of the housing, typically for inserting a non-sterile component, such as a battery, battery assembly, and/or motor.

In some embodiments of the invention, the housing may be configured with two or more ports or access panels. In these embodiments of the invention, a first port in the sleeve or handle allows access to a first portion of the interior of the housing, typically for inserting a first non-sterile component, such as a motor or motor assembly. The second port may be configured and positioned to allow access to a second portion of the interior of the housing, typically for inserting a second non-sterile component, such as a battery or battery pack.

In accordance with the present invention, the housing is also configured to position a motor assembly within a portion of the housing, preferably within a sleeve or hollow handle. As noted in more detail below, the motor assembly may be unsterilized and/or unsterilizable. In most preferred embodiments of the invention, any packaging and medical device should be configured so that the unsterilized motor assembly does not contact a sterilized or sterilizable portion of the package or medical device, as described in more detail below.

In accordance with the present invention, the housing is also configured to position a drive shaft within a portion of the housing. As described in more detail below, the drive shaft is preferably positioned within the housing so that a portion of the drive shaft engages, either directly or indirectly, the motor assembly.

In accordance with the present invention, a medical device includes a motor assembly positioned or configured to be positioned within an internal portion of the housing of the medical device. In the most preferred embodiments of the invention, the motor assembly is re-usable and/or unsterilized or unsterilizable.

A motor assembly of the present invention may be variously configured, as will be readily evident to one skilled in the art. It is intended that the invention should not be limited by the type of motor assembly used, or to any specific arrangement or communication between a motor, a battery, or a drive shaft.

As used herein, motor assembly refers to one or more motors, one or more batteries for powering the motor; one or more various connectors and/or contacts to link individual batteries to each other and to activate a motor, one or more gears or drives or the like for transferring energy in the motor into rotational movement of the drive shaft; or combinations thereof. The batteries may be single use, multiple use, disposable, non-disposable, and/or rechargeable. Various battery assemblies are well known in the art, and it is intended that the invention should not be limited by the type or configuration of the battery assembly. The motor assembly may also consist of the motor itself. In accordance with this aspect of the invention, the motor assembly is typically unsterilized, and preferably re-useable. As used herein, a non-sterile component of the medical device refers to one or motors, one or more batteries, or any combination thereof.

A device according to the invention may be specifically configured to accommodate different power and speed ratings of the motor assembly. The power/speed rating of the motor assembly may be configured for high speed and low torque, low speed and high torque, or variable speed and variable torque, or combinations thereof. Alternatively, using the packaging system and method of the present invention, a surgeon can replace a motor assembly having a first power/speed rating with a motor assembly having a second power/speed rating. For example, a surgeon performing a high speed drilling procedure may wish to briefly perform a low speed grinding procedure. Presently, the surgeon would need to use two different drills to accomplish both tasks. The present invention permits the use of a single drill for both functions by having alternative power/speed configurations that are changeable.

As noted above, the present invention includes a drive shaft positioned in the housing of a medical device, and is adapted to engage a motor. Any structure or series of structures that comprise the contact between the drive shaft and the motor are suitable for use in the present invention. The prior art includes many alternative configurations. It is intended that the present invention should not be limited by the manner in which energy or power is transferred from the motor to the drive shaft.

In some embodiments of the invention, the medical device includes a drive assembly, said drive assembly comprising a drive shaft, a first spline rigidly attached to the drive shaft, a second spline communicating with the first spline, said second spline having an end adapted to align the drive assembly in a nose portion of the housing, and a bushing communicating with the second spline and configured to position the drive assembly in a portion of the housing of the medical device. In some embodiments of the invention, the first spline is rotatable and configured in the housing to prevent axial movement of the first spline. In some embodiments of the invention, the second spline is rotatable and configured in the housing and in relation to the first spline so as to move axially. In some embodiments of the invention, the bushing is non-rotatable and configured in relation to the second spline and the housing to move axially. One of more splines may be inside or outside of the housing, inside a neck or collar portion of the housing, or outside the housing at one or both ends of the drive shaft. In these embodiments of the invention, the drive shaft will typically further include one or more couplings engaged with the drive shaft and for attaching or connecting a tool to the drive shaft. The coupling may be positioned on the drive shaft inside or outside of the housing. In some embodiments of the invention, the drive shaft includes a distal end configured to function as a tool, e.g., the end configured as a drill bit, or shaped with cutting teeth, or the like.

In some surgical or medical procedures, it may be desirable to use separate devices having different torque and speed ratings, or to exchange a component having a first rating with a component having a second rating. For example, it may be desirable to provide a first medical device having a low speed rating, e.g., from about 0 to about 1000 rpm, preferably up to about 400 rpm, and a second medical device having a high speed rating, e.g., from about 5000 to about 20,000 rpm. Alternatively, the medical device may be provided with a first motor, battery, and/or gear assembly having a low speed rating; a high speed rating may be achieved by exchanging the first assembly with a second motor assembly having a high speed rating.

In accordance with the invention, the drive shaft may be variously configured. Typically the drive shaft will be cylindrical and rotatable. The end of the drive shaft distal from the motor, i.e., the tool end, may or may not extend beyond the housing of the medical device. In some embodiments of the invention, it may be desirable for the end near the motor to extend up to or through the housing. In some embodiments of the invention, the drive shaft may include a toothed edge configured to engage a lever or the like. In a preferred embodiment, this configuration of the drive shaft functions as a pawl and ratchet system to permit incremental proximate to distal movement of the drive shaft in the housing. In some embodiments of the invention, the drive shaft may be hollow, preferably to allow the drive shaft to function as a channel through or partly through the housing. For example, a hollow drive shaft could function as a coring instrument. In another example, it may be desirable to allow the operator to pass a wire or some other medical device from the proximal to the distal end of the drive shaft.

In accordance with the present invention, communication between the motor assembly and the drive shaft may be configured to achieve rotational, reciprocal, or oscillating movement in the drive shaft. Alternatively, the neck portion of the housing may be removeable. In accordance with an aspect of the invention, different removeable neck portions may include different internal structures or elements configured to impart a pre-determined motion to the drive shaft. For example, a first neck portion may include structures to impart a rotational movement to the drive shaft, a second neck portion for reciprocal movement, etc.

In some embodiments of the invention, the drive shaft moves rotationally. In other embodiments of the invention, the drive shaft may move rotationally and axially (proximally and/or distally in relation to the end of the housing containing the motor). In some embodiments of the invention it may be very important to maintain concentric movement of the rotating drive shaft. For example, non-concentric movement of the drive shaft will produce a wobble in the distal end of the drive shaft; such a wobble would be highly undesirable in some surgical procedures that require a high degree of precision.

According to the invention, the distal end of the drive shaft may be variously configured or adapted to engage a coupler and/or shaped to function as a tool. For example, the end may hollow and cylindrical to function as a coring device, may have one or more cutting edges for making holes (e.g., function as a drill bit), or one or more cutting edges for burring. It is intended that the invention should not be limited by the type of tooling incorporated in the end of the drive shaft.

As described in the specific embodiments below, a medical device according to the invention may include one or more elements configured to position the drive shaft in the housing, said structures permitting axial and/or rotational movement of the drive shaft. Exemplary elements include but are not limited to a bushing, a spline, a guide, a bearing, a clutch, and the like. Typically, these elements will have a shaped or annular bore configured to the shape of the drive shaft and adapted to seat the drive shaft. In some embodiments of the invention, one or more of these various structures, alone or in combination, may be configured to prevent the drive shaft from moving in any direction other than rotationally. In other embodiments of the invention, it may be desirable to permit the drive shaft assembly to move both rotationally and axially. For example, in a wire driver, a medical device designed to implant a wire, one or more structures, typically including a collet, permit the wire to advance incrementally.

In some embodiments of the invention, an end of the second spline may be variously configured to receive a variety of coupling elements.

In some embodiments of the invention, a portion of the second spline may be adapted to communicate with an actuator assembly.

In some embodiments of the invention, the medical device may include one or more couplers. Typically, the couplers will be multi-functional, i.e., include portions suitable for engaging the coupler to the drive shaft and include portions suitable for engaging a second coupler or a tool. The couplers may be configured to attach a bit or the like to the medical device, or may be configured to attach another coupler. Exemplary couplers for attaching a bit or the like include but are not limited to a chuck, such as a Jacobs chuck, a removable or non-removable chuck, either keyed or keyless or quick-release, either disposable or non-disposable, or combinations thereof; a removable or non-removable collet, either disposable or non-disposable, or combinations thereof. Exemplary couplers for coupling other couplers include but are not limited to, some collets and a keyless chuck.

In a preferred embodiment of the invention, the coupler is an internal coupler, i.e., enclosed within the housing of the medical device or a portion of the housing. Such a position may be beneficial to the overall operation of the medical device by improving concentric movement of the drive shaft, by protecting tissue from moving parts of the medical device, or by protecting a gloved operator from damaging the glove.

A device according to the present invention may also include an easily adaptable coupling mechanism for attaching bits and the like to the device, for changing bits, for increasing or maintaining the sensitivity of the device, and for decreasing the cost of the device. In combination, these attributes contribute to overall improved ease of use, increased simplicity in several critical surgical functions, and decreased waste and cost.

The present invention may also include a packaging apparatus and system for aseptically assembling a sterile device and a non-sterile component of the device. In preferred embodiments of the invention, the assembled device is suitable for use in a sterile environment.

The present invention is also an apparatus for packaging and sterilizing a device comprising an enclosure that maintains a device in a sterile condition and permits insertion of a non-sterile component of the device into a portion of the sterile device while maintaining the device's sterile condition.

The present invention may also include methods for packaging a device having a sterile component and a non-sterile component. An embodiment of the invention includes positioning the device in a package adapted to receive the device and adapted to permit access to a sleeve in the device, sterilizing the device in the package, and inserting a non-sterilized component of the device into the sleeve using an access port in the package. In preferred embodiments of the invention, the method also includes sealing the port prior to sterilizing the package containing the device.

The present invention may also include a system and method for assembling a device having a sterile component and a non-sterile component. An exemplary apparatus and method includes a non-sterile motor assembly positioned into a sterile drill without contaminating the drill. Embodiments of the system and method include a packaging system configured to receive a sterilized medical device having a sleeve or the like for enclosing a non-sterile component of the device. The packaging system and method may involve access through the packaging system into the sleeve without contaminating the previously sterilized portions of the device.

In another embodiment of the invention, the apparatus comprises a sterile device in sterile packaging, with the sterile device comprising a sleeve or the like having a cover in an open position, said sleeve having an inside portion adapted to communicate with a port in the sterile packaging. The port permits insertion of a non-sterile component, such as a battery, battery pack, and/or motor, into the sleeve of the sterile device. In some embodiments of the invention, the port may be covered with a sterilizable cover; in other embodiments, the entire sterile package or a portion of the package that includes the port may be enclosed in an outer enclosure, such as a bag or the like.

The present invention provides a method for placing a non-sterile medical device component into a sterile sleeve so that the sterilized medical device can be used in a medical procedure that requires aseptic technique. More particularly, the present invention comprises an outer enclosure that contains the sterile sleeve and holds it so that only the inside of the sterile sleeve can be accessed through a separate opening in the packaging. The non-sterile component can be placed into the sterilized device by an un-gloved (non-sterile) person. The outside of the sterile sleeve cannot be inadvertently contaminated during the insertion procedure. After the non-sterile component has been inserted, the invention can be opened and the sterile sleeve, with the non-sterile medical device contained within, can be removed by a person wearing sterile gloves.

The present invention also permits the assembly of a non-sterile component with a sterile component; the two parts, when assembled, form a complete medical device which is sterile on its outer surface. An example of a two component medical device is a disposable surgical drill. Typically, an apparatus such as a disposable drill is packaged as a sterile, single use, disposable medical device. A motor, typically re-usable and non-sterile, must be inserted into the handle of the drill to make it functional. The present invention permits insertion of the non-sterile motor into the sterile drill without any chance of contamination of the outside of the sterile drill. The invention can then be opened and the drill removed by a gloved operator. A hatch door on the sterile drill closes the handle portion and protects from any contact with the non-sterile motor contained inside. At the end of the procedure, the hatch door is opened and the drill placed back in the invention. The invention is closed, thereby containing all surfaces of the drill which have come in contact with the patient. The motor is then removed and retained for future use. The invention with the contaminated drill can then be discarded.

The present invention also includes a method for providing an operable surgical device comprising a sterile device and a non-sterilized component. The method comprises positioning a device in a package adapted to receive the device and having a sealable port, sterilizing the package and device, inserting a non-sterile component of the device into the device through the port, opening the package, closing a cap over the non-sterile component This process results in an operable device suitable for use in a sterile environment and having both sterile and non-sterile components.

A package according to the invention may include one or more of the following components: a drill, a drill having an attached disposable chuck, a drill having a disposable quick-release chuck, a drill having a disposable collet, one or more chucks (keyed or keyless), one or more quick-release chucks, one or more collets, a motor, one or more batteries, a motor/battery assembly, and one or more drill bits.

A package according to the invention may be variously configured, preferably to accommodate or position one or more of the components noted above.

An assembled device according to the invention typically refers to a fully operational device. For example, a typical device would not include a battery and/or a motor, both of which should not be sterilized for reasons noted above. A fully operational device would include all of the elements sufficient for the device to function.

An assembled device according to the invention is suitable for use in a sterile field. A sterile field refers to any location where contamination is undesirable and/or where contamination should or must be maintained at a minimum level. Exemplary environments include but are not limited to medical (e.g., surgery rooms) and electrical (electronics assembly rooms) environments. In a preferred embodiment of the invention, the sterile field is a surgical theater or operating room.

A package or packaging system of the invention may be adapted to receive a device. As used herein adapted to receive refers to forms and sub-structures of the package intended to contact the device and hold the device in position in the package. For example, the package may be molded to conform with the shape of the device. Alternatively, the package may include inserts of the like that have been molded or shaped to conform to the shape of the device. Typical inserts include one or more pieces of foam or the like shaped to conform to the shape of the device or a portion of the device. In some embodiments of the invention, adapted to receive the device includes one or more structures that engage a portion of the device, such as a sleeve. In these embodiments of the invention, these structures engage or form an integral portion of a port in the package.

In accordance with the present invention, the device includes one or more structures adapted to receive a non-sterile component of the functional device. As noted above, the non-sterile component typically refers to one of more batteries and/or a motor. As used herein, adapted to receive a non-sterile component refers to one or more structures in the device suitable for enclosing the non-sterile component and sealing the non-sterile component form the sterile component(s) until the sterility of the functional device is no longer a concern. Exemplary structures suitable for enclosing the non-sterile component include but are not limited to a sleeve, housing, conduit, hollow member, or any other suitable enclosure. In a preferred embodiment of the invention, the sterile device includes a hollow handle, the inside portion of which may be adapted to receive one or more batteries and/or a motor.

A device according to the present invention may also include structures that are configured for and/or are adapted to permit access to the device structure suitable for enclosing the non-sterile component. In a preferred embodiment of the invention, this structure is a port in the package. In a most preferred embodiment of the invention, the port is sealable or coverable. Typical structures for sealing or covering the port include but are not limited to a removable or penetrable seal, an outer package that covers the port, or an outer package that covers an inner package having a port.

As noted above, the invention includes any sterile or sterilizable device that typically require the use or insertion of a non-sterilized component. Non-sterilized components include but are not limited to a battery, a battery pack, a motor, a motor/battery assembly, and the like.

In another embodiment of the invention, the apparatus and method may also include re-packaging a used (e.g., non-sterile and/or contaminated with body fluid) device in the packaging, closing the packaging, and disposing of the package containing the used device. Typically, the disposal process will include disposing of the non-re-useable portions of the device. For example, in preferred embodiments of this aspect of the invention, a battery pack and/or motor may be removed from the device prior to re-packaging. In this way, at the end of the surgical procedure, all patient contact elements may be disposed in a manner that reduces the possibility of contact with contaminated components used during the surgery. This combination of sterile and non-sterile components strikes a balance between the desirability of disposable medical instruments and devices with the reality of medical cost containment.

As used herein, adapted for communication, communicating, or similar terms refer to any means, structures, or methods for establishing contact between one element of the system and another element. These structures and/or means are well known by practitioners in the art. Exemplary structures are shown in the Figures. For example, a package may include one or more structures that are complementary to the shape of the device and are intended to position the device in a pre-determined place in the package. In a preferred embodiment of the invention, a port in the package may include, be adjacent to, or communicate with a tab or mount or the like that engages a portion of the sleeve on the device. As used herein, connector refers to any structure used to form a joint or to join itself to another piece. Typical connections include but are not limited to mating connections, such as Luer-type, screw-type, friction-type, or connectors that are bonded together.

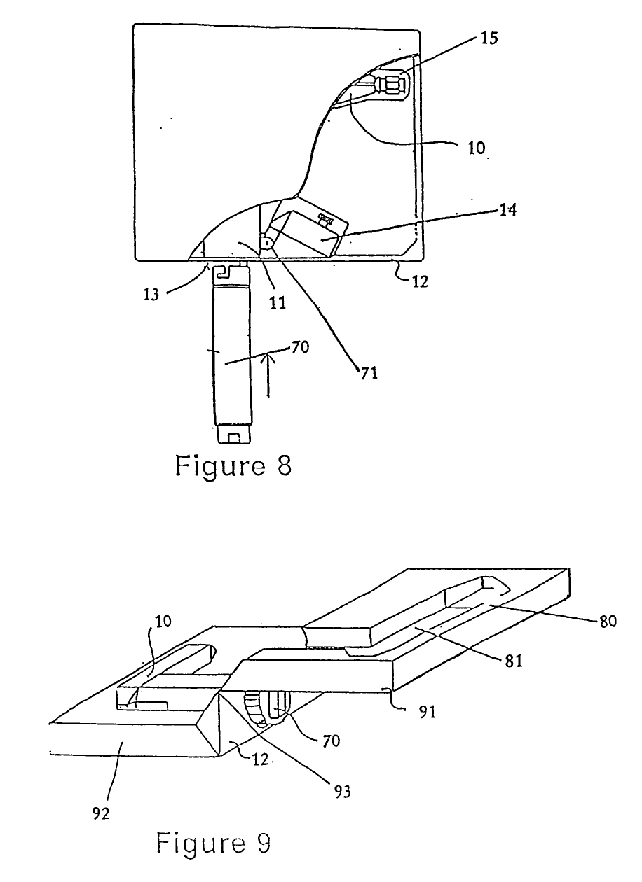

The present invention, exemplary embodiments of which are shown in the Figures, is a medical device 1 adapted and configured for orthopedic use. The medical device includes a sterile housing 10 having a sleeve 11 and a sleeve cover 14, both of which are described in more detail below. The housing and sleeve may be variously configured. The illustrated embodiment of an exemplary medical device includes a housing and sleeve in a pistol-grip configuration. A motor assembly 70 (illustrated in FIGS. 8 and 9) below, engages a proximal end of drive shaft 20. The sleeve 11 is configured to receive a non-sterile motor assembly. In a preferred embodiment of the invention, the housing 10 includes a neck portion 10a, typically configured to seat a nose collar 31. The housing encloses a drive shaft 20 that may be positioned in the housing by one or more internal or external bushings 28. Some embodiments of the invention include one or more switches for activating the device, for rotating the drive shaft in a first direction, and/or for rotating the drive shaft in a second direction, e.g., a first switch 17 (e.g., a forward switch) and a second switch 18 (e.g., a reverse switch).

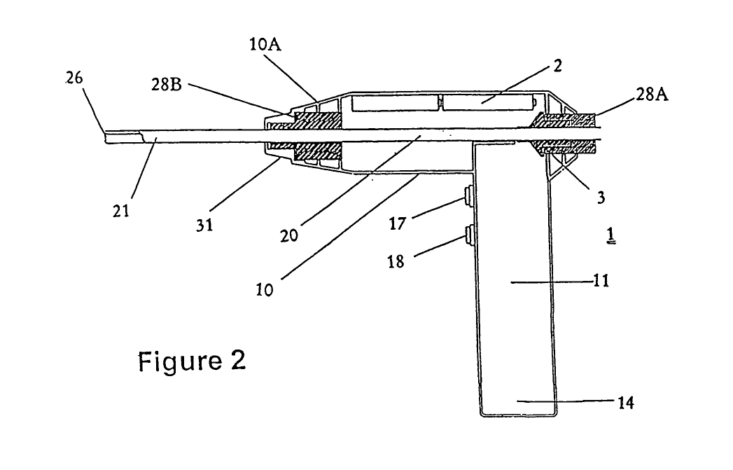

FIG. 2 illustrates the internal components of a typical medical device 1 according to the invention. A motor (not shown) communicating with one or more batteries 2 drive one or more gears 3 for rotating drive shaft 20. Drive shaft 20 is positioned in the housing 10 with one or more bushings. The embodiment illustrated in FIG. 2 includes a proximal bushing 28a and a distal bushing 28b. In a preferred embodiment of the invention, bushings 28, 28a, and 28b do not rotate. In some embodiments of the invention, drive shaft 20 extends through a bore in neck portion 10a, and a bore or opening in neck collar 31. A distal portion 21 of drive shaft 20 may be adapted to receive a coupler or the like, and/or may be configured as a tool. In the embodiment shown in FIG. 2, distal end 26 of drive shaft 20 is itself a tool, configured as a coring tool, i.e., including a hollow shaft and a cutting edge.

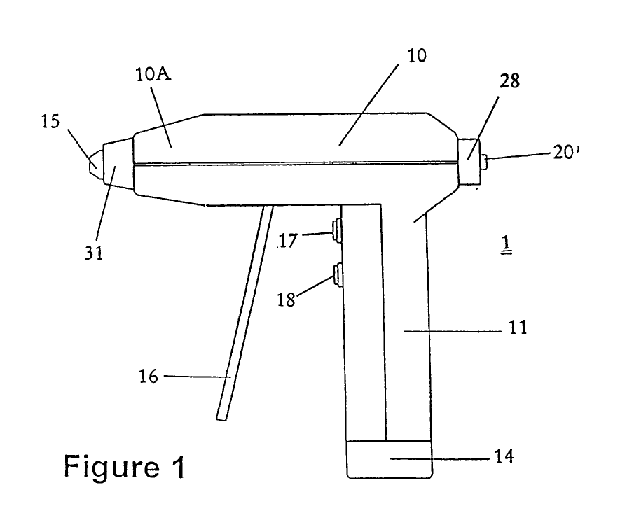

As shown in FIG. 1, some embodiments of the invention include at least one coupling element 15 positioned within the neck portion 10a, the housing 10, the nose collar 31, and/or outside of housing 10. For the embodiments of the invention in which the coupling 15 is a collet, the medical device may include an actuator 16 that communicates with the collet to releasably engage a medical wire or the like.

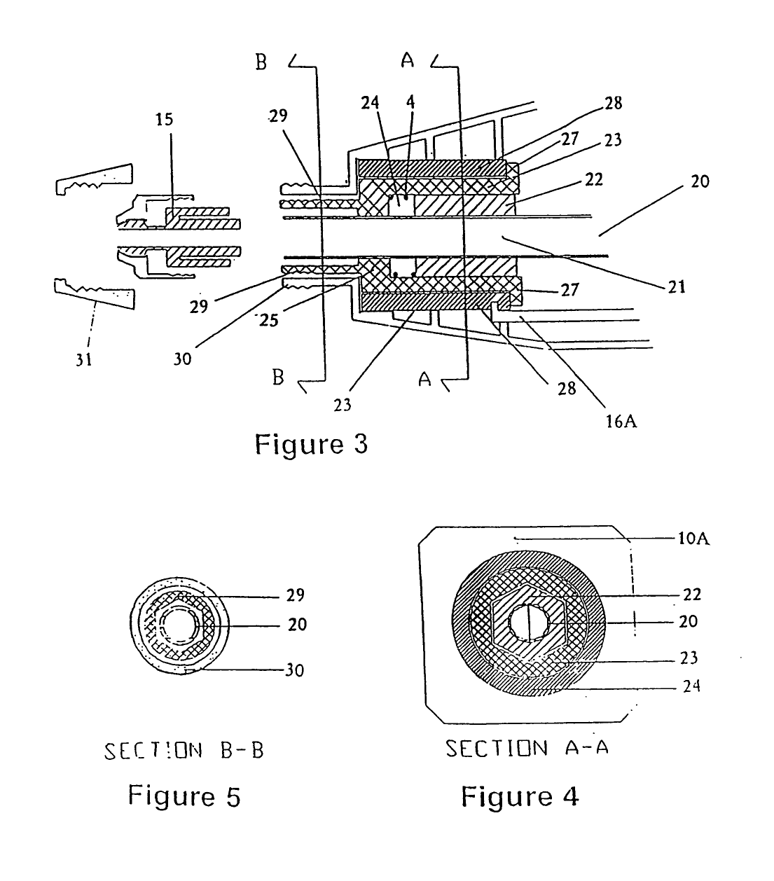

FIGS. 3-5 illustrates an alternative neck portion 10a of a medical device 10 according to the invention, typically, a configuration suitable for use with an internal coupler 15. A distal portion 21 of drive shaft 20 engages first spline 22. First spline 22 may be variously configured and includes a bore through which drive shaft passes. In a preferred embodiment of the invention, first spline 22 matingly engages drive shaft 20 and rotates in concert with the rotational movement of drive shaft 20. A portion of first spline 22 is adapted and configured to engage a portion of a second spline 23.

The configuration of first spline 22 in relation to second spline 23 preferably includes a cavity 24 or the like that encloses spring 4 or the like, and allows proximal and distal movement of second spline 23 in relation to first spline 22. Second spline 23 also may include a portion 25 configured to moveably engage and position a distal portion 21 of drive shaft 20. In a preferred embodiment of the invention, second spline 23 is capable of moving rotationally. Second spline 23 may also include one or more first alignment elements 27 adapted to engage a bushing 28 or the like positioned within a cavity in the housing. In a preferred embodiment of the invention, second alignment element 29 may also be adapted to matingly engage and seat a coupling element 15. In the illustrated embodiment, second alignment element 29 includes threads to engage the corresponding portions of the coupler.

Bushing 28 may also be configured to engage a portion 16a of actuator assembly 16. In the exemplary embodiment shown in FIG. 3, actuator assembly 16, first spline 22, second spline 23, bushing 28, space 24, spring 4, and neck 30 communicate together and function to permit axial movement of second spline 23 and bushing 28 within the housing. Preferably, drive shaft 20 and neck 30 do not move axially. In a preferred embodiment of the invention, first spline 22, second spline 23, bushing 28, and neck 30 are configured to function in combination to position the drive assembly in the nose portion 19 of housing 11. In a preferred embodiment of the invention, first spline 22, second spline 23, bushing 28, and neck 30 are configured to function in combination to position the drive assembly in the nose portion 19 of housing 11.

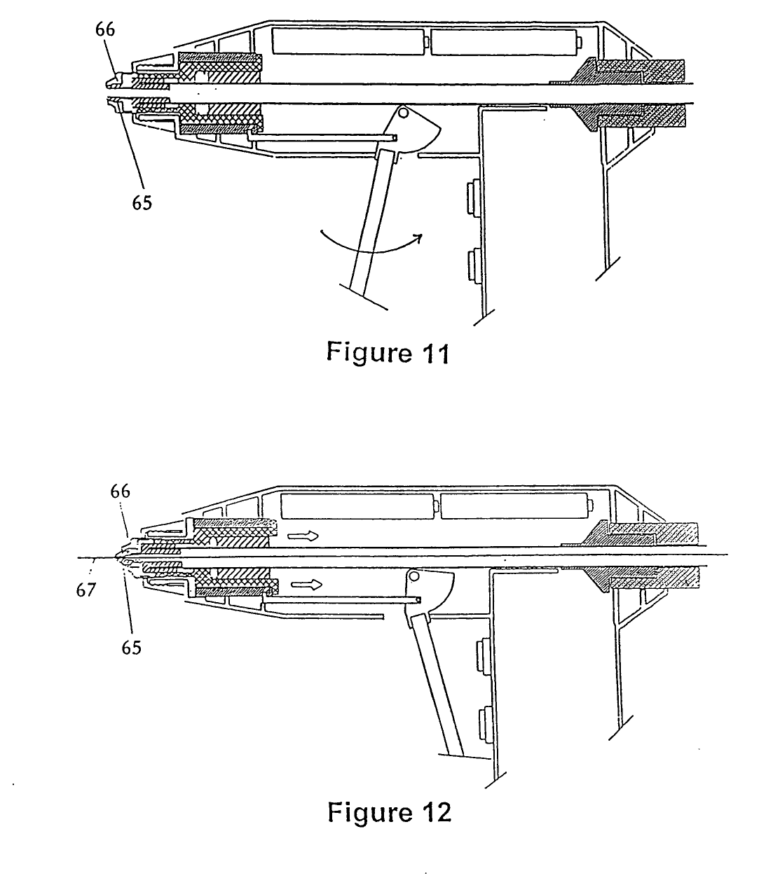

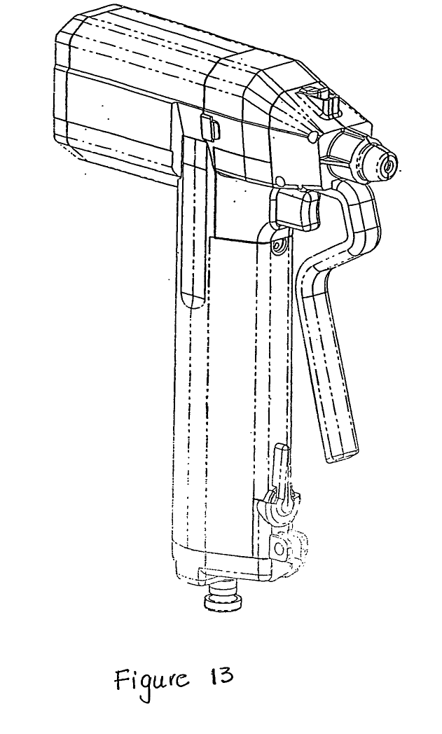

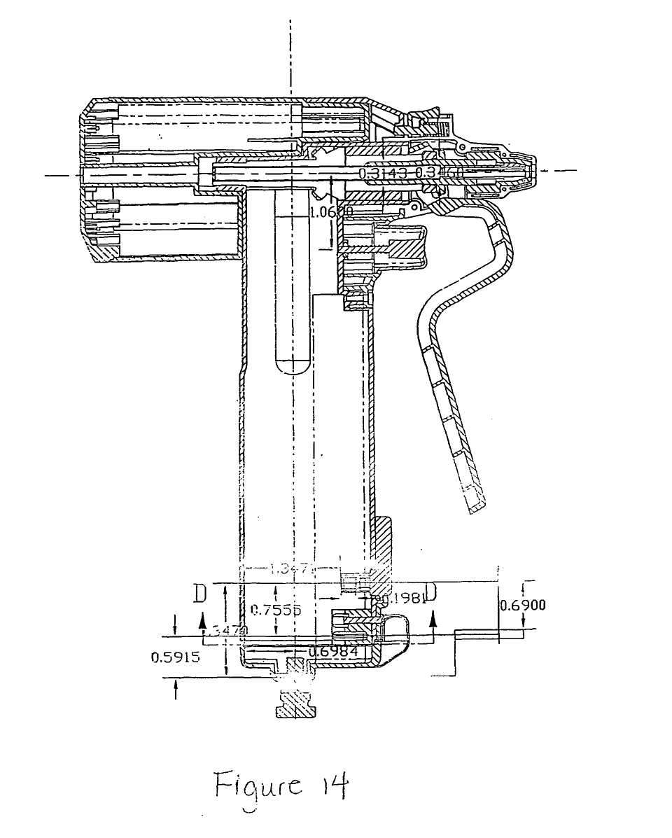

As shown in FIG. 3, the neck portion 10a of the medical device may include a neck 30 configured to align second alignment element 29 of second spline 23 in the nose portion of the housing. Further, neck 30 may be configured to engage a neck collar 31. Neck 30 may engage neck collar 31 in any suitable manner, including but not limited to a friction fit, screw threads, or a Luer-type connection. The illustrated embodiment shows a threaded connection. Neck cover 31 at least one bore 32 configured to allow a coupler, bit, collet, or wire to pass therethrough. The exemplary embodiment shown in FIGS. 11 and 12 is an exemplary configuration of a medical device 10 adapted to install a medical wire, such as a K wire or a fixation wire.

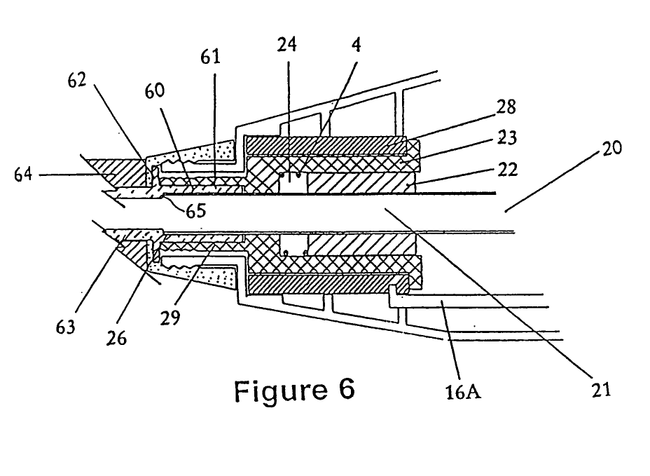

FIG. 6 illustrates another alternative neck portion 10a of a medical device 10 according to the invention, typically, a configuration suitable for use with an external coupler 15. In this embodiment of the invention, the shape, communication, and function of distal portion 21, drive shaft 20, first spline 22, second spline 23, cavity 24, spring 4, portion 25, first alignment element 27, bushing 28, and second alignment element 29 is the same as described above for FIG. 3. In this embodiment of the invention, second alignment element 29 of second spline 23 is adapted to engage or receive a coupler adapter or coupler collar 60. A proximal end 61 is configured to matingly engage the distal portion 21 of drive shaft 20. In a preferred embodiment of the invention, coupler collar 60 includes a seat 65 configured to engage the distal end 26 of drive shaft 20, and even more preferably to matingly engage the drive shaft to reduce or prevent concentric movement. In a preferred embodiment of the invention, coupler collar 60 also includes a shoulder 62 configured to be aligned between the end of second spline 23 and neck collar 31. Preferably, the neck collar is configured to prevent the coupler from disengaging the medical device during operation. In a typical configuration of this embodiment, coupler collar 60 may also include a sleeve 63 or the like for communicating with and engaging a coupler 64, such as a chuck.

In a preferred embodiment of the invention, various components or structures of the medical device are formed of materials adapted to promote increases sensitivity or feel for the operator. For example, the sensitivity of the medical device may be related to the tip of a bit transmitting relative or subjective information to the operator regarding the location of the tip of the bit in tissues of varying densities and/or resistance. This aspect of the invention is in direct contrast to some devices in the prior art that contain heavier components designed to withstand sterilization. An object of the present invention is to provide a medical device formed of components that contribute to an overall increase in sensitivity of the medical device.

FIGS. 11 and 12 illustrate the function of a medical device 10 substantially as described for FIG. 3. The illustrated embodiment is specifically designed for use with a coupler assembly 15 that comprises a collet. In this embodiment of the invention, the coupler assembly includes a displaceable resilient tip 65 in communication with the distal end 26 and/or distal portion 21 of drive shaft 20. The coupler assembly 15 also includes a flange 66 configured to matingly engage alignment member 29. Although these structures may be variously configured, the illustrated embodiment shows a stationary flange 66 in relation to a moveable resilient tip 65. By moving the actuator to a closed position (shown in FIG. 12), flange 66 moves axially in relation to resilient tip 65, thereby closing or pinching tip 65 against a wire 67 or the like.

As noted above, a distal end of the drive shaft may include or may be adapted to receive a tool. As used herein a tool includes but is not limited to drills, taps, reamers, probes, sounders, corers, burrs, various extension shafts, countersinks, shannons, lindemanns, eggs, pears, ovals, fissures, helicoidal rasps. Other tools are well known in the art and may be configured for use with the medical device 10 of the present invention.

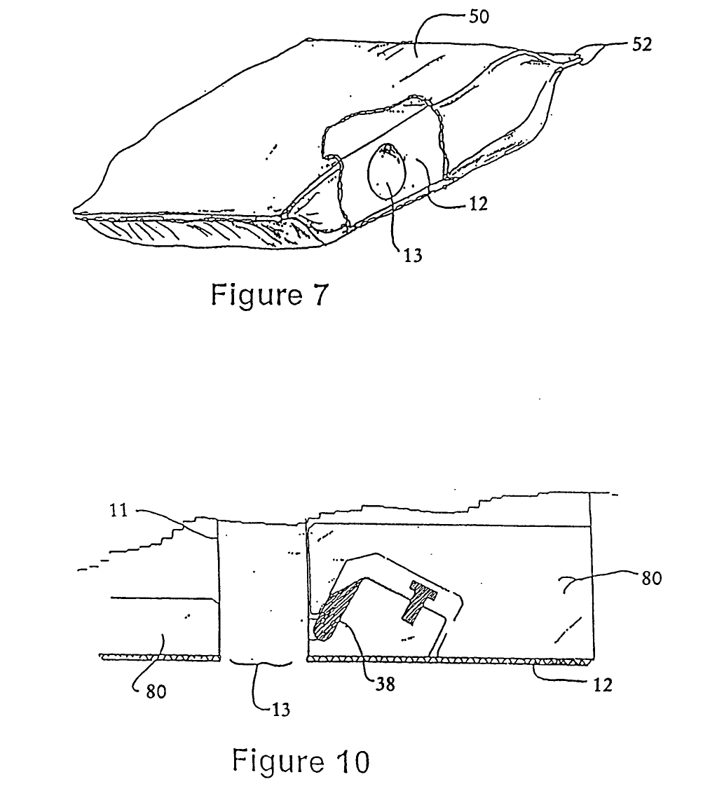

The present invention also includes a package 12 suitable for enclosing a device 10 having a portion 11 suitable for engaging and/or enclosing a non-sterilized component of the device. In a preferred embodiment of the invention, package 12 includes a sealable port 13. In a most preferred embodiment of the invention, device 10 also includes a closable cover 14 for capping or closing portion 11. FIGS. 7-10 show exemplary embodiments of this aspect of the invention.

FIGS. 7-10 show packaging or enclosure 12 for positioning and maintaining a medical device 10 in a sterile condition until the medical device is ready for use. Enclosure 12 may be constructed of any material compatible with use in a sterile environment, and capable of withstanding sterilization. A wide variety of these containers are already known in the art. For example, these enclosures may be made from plasticized polyvinyl chloride, e.g. PVC plasticized with dioctylphthalate, diethylhexylphthalate, or trioctyltrimellitate; polyolefin, polyurethane, polyester, and/or polycarbonate. It is intended that the present invention is not limited by the type of material used to construct the container.

Enclosure 12 may be of any shape and size. One skilled in the art will recognize that the size and shape of device 10 will typically dictate the size and shape of enclosure 12 as well as various optional inserts positioned within the enclosure 12. Enclosure 12 may comprise one or more parts, sections, segments, or the like; preferably enclosure 12 is openable, closable, and/or sealable. In the embodiment of the invention shown in FIG. 9, enclosure 12 may comprise an upper section 91 and a lower section 92 molded or joined by a hinge 93 region. Section 91 and section 92 matingly engage when in a closed position.

Enclosure 12 may be adapted to receive medical device 10. Preferably enclosure 12 is adapted by forming portions of the enclosure to engage and/or secure device 10 in place. One skilled in the art will recognize that a variety of structures, materials, and systems may be employed to position device 10 in enclosure 12. In a preferred embodiment of the invention, an interior section of enclosure 12 is molded to the shape of device 10. For example, FIG. 9 shows a void or cavity 81 adapted to receive device 10. Alternatively, enclosure 12 may include one or more inserts 80 configured to be positioned within enclosure 12 and configured to receive or engage device 10. As illustrated in FIG. 9, insert 80 may be shaped or configured with one or more cavities to matingly engage or secure device 10. One or more inserts 80 may be formed of any material that is sterilizable, and is preferably formed of a foam or spongy material.

As shown in FIG. 10, enclosure 12 comprises a port 13 positioned adjacent to, contiguous with, or proximate an inside portion of sleeve 11 of device 10. In the most preferred embodiments of the invention, port 13 is sealable. Any structure or means for sealing port 13 may be used in accordance with the present invention. Exemplary structures include but are not limited to an outer or second enclosure 50 that encloses package or first enclosure 12 including port 13 (see FIG. 7), or a covering or seal or the like for sealing port 13 and/or the area adjacent to port 13. A preferred embodiment of the invention includes both a cover over port 13 and a second enclosure 50 surrounding first enclosure 12.

Upper section 91 and lower section 92 of enclosure 12 may be sealed with a gasket or removable tape along the seam between upper section 91 and lower section 92. Further, port 13 of enclosure 12 may be sealed along the edge of port 13 using cover or the like. Cover may be a removable strip or the like or may be penetratable membrane or the like. When removable seal is removed or penetrated, access to the inside of sleeve 11 of device 10 is permitted. In a preferred embodiment of the invention, the combination of the removable tape and removable cover prevents contamination of the device 10 positioned in enclosure 12 until it is ready for use.

Outer enclosure or pouch 50 prevents contamination of the inside of the device contained within enclosure 12 until the device is needed. Pouch 50 may be constructed of any material typically used to protect sterile surgical and medical instruments from contamination. The pouch 50 is preferably sealable and/or completely sealed along its edges. In FIG. 7 pouch 50 is shown partly cut away to reveal enclosure 12 and port 13 contained within.

Enclosure 12 may comprise an integral structure, two or more mated and/or matable sections, or combinations thereof. A preferred embodiment of the invention comprises enclosure 12 comprising upper 91 and lower 92 sections (see FIGS. 8 and 9). FIG. 8 shows enclosure 12 in its closed position. A releasable capture mechanism, not shown, keeps the upper section 91 and the lower section 92 matingly engaged until the enclosed device 10 is needed. In the illustrated embodiment, enclosure 12 is openable, preferably using a hinge or the like to provide access to the device 10 contained within.

In the embodiments of the invention shown in FIG. 8 and 10, sleeve 11 of device 10 is positioned in alignment with, adjacent to, and/or engaged with port 13. In a most preferred embodiment of the invention, port 13 permits access to an inner portion of sleeve 11 while enclosure 12 is closed. In the illustrated embodiments, sleeve 11 is a hollow handle of device 10. As shown, sleeve 11 of device 10 is positioned in the sterile enclosure 12 so that an open end of the handle is aligned with port 13 in the side of the enclosure 12. One skilled in the art will readily recognize that a variety of structures and means may be used to properly position the open end of sleeve 11 in relation to port 13. FIG. 8 illustrates an exemplary configuration in which a cavity 15, not adjacent port 13, positions sleeve 11 adjacent port 13. FIG. 10 shows an exemplary configuration is which one or more inserts 80 that position sleeve 11 adjacent port 13.

FIG. 10 shows an exemplary configuration is which port 13 and/or enclosure 12 include a seat, guide, tab, or the like that aligns or matingly engages an inner portion of sleeve 11 to position sleeve 11 in relation to port 13. The present invention includes using one or more of the these configurations to position the inside of sleeve 11 in relation to port 13.

The present invention also includes a method for combining a non-sterile component of a device with a sterile component of the device while maintaining the sterility of the sterile component. Exemplary structures that illustrate the methods of the invention are shown in FIGS. 7-10.

As is well known to practitioners in the art, the protocols for maintaining the sterility of a device 10 until it is operable and/or ready for use are varied. Typically, these protocols involve a single technician performing certain actions while not sterile (in medical parlance, un-gloved) and other actions while sterile (e.g., gloved), or the coordinated actions between a sterile technician and a non-sterile technician. The essence of these actions is that a non-sterile technician should not touch a sterile device or enter a sterile field, and likewise, a sterile technician should not touch a non-sterile device. The various embodiments of the present invention accommodate all of these protocols.

In accordance with the apparatus and method of the present invention, the portion of the device 10 that needs to be sterilized is placed in enclosure 12. At this point in the process, port 13 of enclosure 12 may be open or sealed, but is preferably sealed with a covering or tape. Enclosure 12 is then closed and preferably sealed, as described above. The closed enclosure 12 may then be sterilized, but it is preferable to place the closed enclosure 12 in an outer pouch 50 prior to sterilization. If port 13 does not have a seal or cover, enclosure 12 must be placed in an outer pouch 50 prior to sterilization. Enclosure 12 containing a portion of device 10 may then be sterilized.

Once sterilization is complete pouch 50 containing enclosure 12 and device 10 may be stored until ready for use. When ready for use, a non-sterile technician opens pouch 50 in a controlled environment, such as a procedure room, a clean room, or an operating room. The non-sterile technician may then remove enclosure 12 and insert a the non-sterile component 70 of device 10 into the sleeve 11 of device 10. The non-sterile component 70 is one or more elements needed to make device 10 functional or operable, but which may be deleteriously affected by exposure to sterilizing agents. Non-sterile components include but are not limited to one or more batteries, one or more motors, a battery pack, or combinations thereof.

In some embodiments of the invention, the non-sterile technician will then remove cover from port 13 and slide non-sterile component 70 into sleeve 11. In other embodiments of the invention, the non-sterile technician will push the non-sterile component through a penetratable seal covering port 13. These actions are illustrated in FIG. 8. In this embodiment of the invention, the top of sterile enclosure 12 is shown partially cut away, revealing the enclosed device 10, such as a sterile surgical drill. It should be noted that the sterile enclosure 12 can be handled by a non-gloved operator without contaminating the outside of enclosed sterile device 10 because the portion of enclosure 12 covering device 10 is still closed or sealed. The same non-gloved operator can also insert the non-sterile component 70 into the sterile device 10. In a preferred embodiment, the non-sterile component 70 may be locked into position in the sterile device 10.

The non-sterile technician then opens enclosure 10, as is shown in FIG. 9. The upper section 91 of enclosure 12 has been folded back to provide access to sterile device 10. At this point, a sterile or gloved technician may touch device 10, now containing a non-sterile component. The sterile technician should limit contact to only the inside of the sterile package 12 and the sterile device 10.

In some embodiments of the invention, device 10 is now functional or operable by a sterile or gloved person. In alternative and preferred embodiments of the invention, device 10 or sleeve 11 includes a cover 14, sterilized at the same time that device 10 is sterilized, that can then be closed over any protruding or uncovered portion of non-sterile component 70. One skilled in the art will recognize that cover 14 can seal sleeve 11 using a variety of structures and mechanisms. Exemplary structures include but are not limited to a friction fit or snap fit, or a spring-loaded assembly between cover 14 and sleeve 11, and/or a hinged arrangement between cover 14 and sleeve 11. The preferred embodiment for this aspect of the invention includes a hinge. In a most preferred embodiment of the invention, cover 14 includes a thumb screw or the like for ascertaining that the cover 14 remains closed or sealed over non-sterile component 70.

The sterile or gloved technician may then close cover 14 and tighten the closure screw. Device 10 containing non-sterile component 70 is now fully operational and suitable for handling and operating by a sterile or gloved person.

After use, the non-sterile component can be removed from the device 10 and reused one or more times.

In a further embodiment of the invention, once the operator has finished using device 10, it may be re-packaged in enclosure 12 to insure that other are protected from the now-contaminated device. In this embodiment of the invention, the intent is to retain the contamination inside the enclosure, close the enclosure, and transport the closed enclosure containing the contaminated device to a treatment center. Typically, any re-useable components of the device, or components that should not be sterilized, are separated from the device. The remainder of the device, e.g., the sleeve, handle, and/or housing, may be disposed or may be re-sterilized prior to use again.

Although the present invention has been described in terms of a particular preferred embodiments, it is not limited to those embodiments. Alternative embodiments, examples, and modifications which would still be encompassed by the invention may be made by those skilled in the art, particularly in light of the foregoing teachings.

1. A cordless medical apparatus suitable for use in orthopedic medical applications comprising a disposable sterile housing having a sleeve, said sleeve being configured to receive an unsterilized motor assembly.

2. The medical apparatus of claim 1 further comprising a drive shaft engaged with said motor, and at least one bushing communicating with said drive shaft.

3. The medical apparatus of claim 2 wherein said drive shaft is configured as a tool.

4. The medical apparatus of claim 1 packaged in a sterile enclosure.

5. An apparatus for aseptically assembling a sterile component and a non-sterile component comprising a sterile sleeve having a non-sterile aperture, and packaging material enclosing said sterile sleeve and having a non-sterile port communicating with said non-sterile aperture.

6. The apparatus of claim 5 wherein said sterile sleeve further comprises a cover for closing said non-sterile aperture.

7. The apparatus of claim 1 wherein the medical device is a drill.

8. A cordless medical apparatus suitable for use in orthopedic medical applications comprising a handle sub-component, said handle sub-component being configured to receive one or more batteries; a collet sub-assembly configured to engage a portion of the handle sub-assembly; and a motor sub-component configured to engage a portion of the handle sub-assembly.

9. The medical device of claim 8 wherein one or more of the sub-components are fluid tight.

10. The medical device of claim 8 wherein one or more of the sub-components are disposable.

11. A method of assembling a device having a non-sterile component and a sterile component comprising providing a device having a sleeve, said sleeve comprising a sterile outer portion and a non-sterile inside portion; providing an enclosure adapted to communicate with said device and adapted to maintain the sterility of the outside portion of said sleeve, said enclosure having a port for communicating with an inside portion of said sleeve; and positioning said non-sterile component in said sleeve by passing said non-sterile component through said port.
